(19) United States
(12) Patent Application Publication
(10) Pub. No.: US 2006/0228420 A1
(43) Pub. Date: Oct. 12, 2006

(54) Pharmaceutical compositions comprising microparticles for delivery into neurons

(75) Inventor: Joanne E. Martin, London (GB)

Correspondence Address: COZEN O' CONNOR , P.C., 1900 MARKET STREET, PHILADELPHIA, PA, 19103, US

(73) Assignee: Queen Mary & Westfield College, London (GB)

(21) Appl. No.: 11/375,827

(22) Filed: Mar. 15, 2006

Related U.S. Application Data
(60) Provisional application No. 60/661,998, filed on Mar. 15, 2005.

(51) Int. Cl. A61K 48/00; A61K 38/17; A61K 31/715; A61K 9/14; C12N 15/87

(52) U.S. Cl. 424/489; 514/44; 514/2; 514/54; 435/459

(57) ABSTRACT

A method is provided for the delivery into a neuron of a microparticle of average particle diameter 0.5 μm containing a pharmaceutically active substance, comprising the administration of said particle to said neuron. Also provided are methods for the treatment of diseases of the nervous system comprising the use of such microparticles containing pharmaceutically active substance.

### CROSS-REFERENCE TO RELATED APPLICATIONS

This application claims priority to U.S. provisional application Ser. No. 60/661,998 filed Mar. 15, 2005, which is incorporated herein by reference in its entirety.

### FIELD OF THE INVENTION

The present invention relates to pharmaceutical compositions that are formulated as microparticles for delivery and uptake into neuronal cells. The compositions also provide for controlled-release or delayed release of the pharmaceutical substances contained in the formulation inside the neuronal cell.

### BACKGROUND OF THE INVENTION

Phagocytosis, the ingestion of particulate ligands whose size exceeds about 0.5 μm, is an evolutionarily conserved process (Greenberg and Grinstein, Curr. Opin Immunol. 14, 136-145 (2002)) and required for a wide variety of specialised biological events (Underhill and Ozinsky, Ann. Review Immunol 20 825-852 (2002)). A variety of distinct processes involving a host of different signalling pathways have been recognised, leading to three coordinated cellular processes; receptor mediated recognition of ligands on particles; cytoskeletal alterations involving the direct polymerisation and rearrangement of actin; recruitment of membrane from internal pools, with endoplasmic reticulum being a major reservoir (Gagnon et al, Cell 110 119-131 (2002)), enabling internalisation of a particle.

Phagocytosis has a role in removal of apoptotic and necrotic cell debris and is required in embryonic development (Henson, P., Proc. Nat'l Acad. Sci. USA, 100, 6295-6296 (2001)). Different ‘types’ of phagocytosis are being recognised, including that which is directly associated with an inflammatory response, and that which is not, and these forms may involve cells with different complements of cell surface receptors, as described in macrophages. Ligands on the apoptotic cell surface, such as phosphatidylserine, can be changed via glycosylation or by alterations in their surface charge (Aderem, A, Cell, 110, 5-8 (2002)), which may signal susceptibility for phagocytosis. Phagocytic receptors that regulate the uptake of apoptotic cells include the scavenger receptors, integrins, lectins, and calreticulin/GP91 complex.

Neurons, whilst known to be capable of endocytosis and pinocytosis, are not regarded as a cell type active in phagocytosis. Neurons are known to be able to take up relatively large molecules during endocytosis, which has been the basis of neuroanatomical tracing studies, and viral uptake is also well studied. Recent retrograde transfer from muscle to spinal motor neuron of a viral vector carrying insulin growth factor-1 has also been demonstrated (Kaspar et al, Science, 301 839-842 (2003)). Traditional teaching is that bulk cellular debris in the nervous system is removed by macrophages derived from microglia. Certain other cell types within the central nervous system, such as retinal pigment epithelial cells, are also known to be phagocytic, particularly during phases of cell death during early development, and a report by Egensperger et al (Dev. Brain. Res. 97 1-8 (1996)) in a description of cellular degeneration in the developing retina, mentions the uptake of cell debris by retinal neurons.

A wide range of non-macrophage cell types are recognised to be capable of phagocytosis—so-called ‘amateur’ phagocytes, but this ability in neurons has not been documented nor been suggested previously.

The treatment of diseases of the nervous system or of neuronal cells presents particular problems in ensuring that the pharmaceutically active substance reaches its intended site of action, is not degraded in the stomach or subject to enterohepatic circulation and degradation, or has unwanted systemic effects on the body of the patient.

### SUMMARY OF EMBODIMENTS

The present invention is directed to methods for the delivery into a neuron of a microparticle of average particle diameter 0.5 μm containing a pharmaceutically active substance, comprising the administration of the particle to the neuron. In some embodiments, the pharmaceutically active substance is a drug. In some embodiments, the pharmaceutically active substance is a protein, nucleic acid, carbohydrate, glycosaminoglycan, proteoglycan, or peptide nucleic acid.

The present invention is also directed to methods for the treatment of a disease or condition of the nervous system comprising the administration of a microparticle of average particle diameter 0.51 μm containing a pharmaceutically active substance effective to treat the disease or condition of the nervous system. In some embodiments, the disease of the nervous system is selected from the group consisting of Cancer, Motor Neuron Disease (MND), Parkinson's disease, Alzheimer's disease, and non-malignant tumours.

The present invention is also directed to a microparticle of average particle diameter 0.5 μm containing a pharmaceutically active substance for use in treatment of a disease or condition of the nervous system.

The present invention is also directed to a unit dosage form of a pharmaceutical composition for the treatment of a disease or condition of the nervous system in which the pharmaceutical composition comprises a plurality of microparticles of average particle diameter 0.5 μm containing a pharmaceutically active substance to treat the disease or condition of the nervous system.

The present invention is also directed to a pharmaceutical composition comprising a plurality of microparticles of average particle diameter 0.5 μm containing a pharmaceutically active substance to treat a disease or condition of the nervous system.

### BRIEF DESCRIPTION OF THE DRAWINGS

The invention will now be further described by way of reference to the following Examples and Figures which are provided for the purposes of illustration only and are not to be construed as being limiting on the invention. Reference is made to a number of Figures in which:

FIG. 1A shows haematoxylin and eosin stained section of lumbar spinal cord in a Loa homozygote E18 mouse showing intraneuronal inclusions with areas of basophilia (arrowhead) within the inclusion body; FIG. 1B shows haematoxylin and eosin stained section of wild type control mouse lumbar spinal cord; FIG. 1C shows ChAT-stained lumbar spinal cord in a Loa homozygote E18 mouse showing positivity of motor neuronal cell bodies and inclusion (arrowhead); FIG. 1D shows Feulgen-stained section of lumbar spinal cord in a Loa homozygote E18 mouse showing positive granules (arrowhead) within intraneuronal inclusions; FIG. 1E shows electron dense debris apparently extending into the neuronal cell body of a lumbar spinal motor neuron from an E18 Loa homozygote mouse showing an intact nucleus and endosomal compartment abnormalities; FIG. 1F shows human cortical neuron containing siderotic granules (dark brown); FIG. 1G shows lipid debris (asterisk, blue stained similar to adjacent myelin (m)) within human dorsal root ganglion cells in Guillain-Barre syndrome, with adjacent uninvolved neurons, including grey granular stained lipofuscin; and FIG. 1H shows myelin figure(m) within the body of a human peripheral nerve axon, from a case of severe axonal neuropathy.

FIG. 2A shows India ink particles within rat basal ganglia neurons (example arrowhead); and FIG. 2B shows microspheres (brightly birefringent bodies) seen within a mouse cerebral cortical neuron.

FIG. 3A scanning electron microscopy images of chick dorsal root ganglion cells in culture incubated with microspheres. From left to right; control dorsal root ganglion neurons; 2.8 micron microspheres shown extracellularly and within glial cells; dorsal root ganglion neuronal cell body containing 7 microspheres, with two seen lying beneath the fractured cell membrane: apoptotic neuron with an attached microsphere; and FIG. 3B shows transmission electron microscopy images of rat spinal motor neurons in culture showing adhesion and uptake of large polyvalent protein complexes labelled with gold-conjugated antibodies and Tf-HRP. Bar 200 nm.

FIG. 4A shows sequence of consecutive images (supplementary video 1) from left to right showing transport towards the cell body of a large volume debris along a neurite in a rat spinal cord neuron in culture (DIV1); and FIG. 4B shows uptake of apoptotic debris (arrowhead) by a rat spinal cord cell in culture (DIV1). The debris originated by the death of a neighbouring cell is transported from a peripheral site towards the soma. Three frames from video 2 (see supplementary data) are shown.

FIG. 5A shows confocal microscopy of rat spinal cord motor neurons (DIV8), stained with an antibody against beta-tubulin to highlight microtubules (in green), after incubation with 2.8 μm microspheres (in red). The series of z-stack images have been processed to assemble a z-section along the dotted line plane (bottom left). Bar, 5 μm. The right panel shows a phase image of the same motor neuron (top) and the z-stack projections of the green and red channels and their merged image. Bar, 20 μm; and FIG. 5B shows confocal microscopy of a rat spinal motor neuron with three internalised microspheres in red and microtubules labelled in green. Description as for panel A. The neuron displays extensive blebbing, which is characteristic of apoptotic cell death. Bars, 5 and 20 μm.

FIG. 6 shows a selection of 1 micron (1 μm) beads labelled with dragon green fluorescence taken up by rat dorsal root ganglion cells.

FIG. 7 shows 3D projection of DRG neurons in culture with dragon green polystyrene microspheres (microtubules in red, nucleus in blue).

FIG. 8 shows microspheres with cell body.

FIG. 9 shows microspheres with cell body.

FIG. 10 shows microspheres with cell body.

FIG. 11 shows a graph showing the addition of cortical neurons cultured (using the dorsal root ganglion cell protocol) with microbead particles added to cell culture dishes, demonstrating lack of toxicity at low concentrations.

### DESCRIPTION OF EMBODIMENTS

It has now been surprisingly found that neuronal cells can phagocytose bulk particles and/or media from their external environment. Thus, the potential now exists for pharmaceutical compositions to be formulated for uptake by neuronal cells thereby overcoming the problems encountered in treating various neuronal cell or nervous system diseases.

According to a first aspect of the invention, there is provided a method for the delivery into a neuron of a microparticle of average particle diameter 0.5 μm containing a pharmaceutically active substance, comprising the administration of said particle to said neuron.

Neurons include, but are not limited to the following cell types, neurons, neuronal cell lines, such as PC12 cells, primary neuronal cell cultures, dorsal root ganglion cells, motor neurons and cortical neurons, or nervous tissue comprising such cells, including nerve, spinal cord or brain.

The microparticle may be present as a population of microparticles in the form of a composition, optionally formulated in a physiologically suitable diluent. The microparticles may be in the form of beads or spheres (i.e. microbeads or microspheres), optionally comprising an internal lumen.

Delivery of the microparticle may be via direct administration of the particle to a cell or cells in culture in vitro, or it may be via direct injection in vivo to a neuron, or it may be via indirect injection into a body cavity or tissue, such as for example in proximity to a neuron or nervous tissue of interest for delivery of the particle. Injection may be into a nerve, the spinal cord, the cerebrospinal fluid, the epidural space, the brain, the intracranial space, the intraocular space.

The microparticle may be composed of any convenient substance. For example, a polymeric material that is preferably inert and non-toxic, or a mixture of polymers and/or copolymers. Examples of such materials include but are not limited to glass, gold, iron, polystyrene, polyethylene, polyester, polytetrafluoroethylene (PTFE), gelatine, alginate, polylactic acid (PLA), monomethoxypolyoxyethylene (MPOE), poly-L-lactide-co-glycolide (PLGA) and polyvinyl alcohol (PVA) or polyorganophosphazenes, derivatized at the phosphorus atoms with phenylalanine ethyl ester and imidazole, or mixtures, co-polymers or polymers thereof. The polymeric material may be crosslinked. Optionally, for ease of use, the particles may be magnetic.

Microsphere preparation can be carried out using any suitable material as described above.

Three common methods of microsphere preparation can be adopted as follows: spray-drying, emulsion/solvent evaporation and emulsion/solvent evaporation-extraction. In many cases, it may prove to be convenient to prepare the microspheres by the “simple” and “double” emulsion methods.

For example, when preparing microparticles, it may be useful to employ a biodegradable material, such as polyorganophosphazenes, derivatized at the phosphorus atoms with phenylalanine ethyl ester and imidazole at desired molar ratios. The polymers can be prepared by substitution of the chloride atoms of polydichlorophosphazene with a phenylalanine ethyl ester-imidazole mixture followed, after 7 or 48 h reaction, by the addition of excess imidazole.

By the appropriate choice of pH and solvent composition of the external phase, the drug substance of choice may be entrapped in the microspheres. The polymer composition dictates the in vitro release rate of the drug from the particles, which may be faster when the microspheres are prepared with the polymer at higher imidazole content. (Veronese et al Journal of Controlled Release, 52, (3), pages 227-237 (1998))

Alternatively, microspheres may be prepared by the emulsion-solvent evaporation process using monomethoxypolyoxyethylene polylactic acid (MPOE-PLA) copolymers as the matrix material and/or the surfactant in order to avoid the use of PVA as the surfactant in the process. In such methods, two series of MPOE-PLA copolymers may be synthesised. The first, with a long and constant length PLA chain can be used as the matrix material, the second, with short PLA chains and different HLB as the surfactant. MPOE-PLA copolymers can be used for the preparation of particles instead of PVA and their use can be extended whenever a biocompatible and bioeliminable surfactant is required for biological or medical applications. (Bouillot et al Pharm Res, 16 (1), 148-154 (1999)).

In one embodiment of the invention, the microspheres may be prepared from poly-L-lactide-co-glycolide (PLGA) and polyvinyl alcohol (PVA), using a process in which a primary emulsion is formed of the PLGA, followed by formation of a secondary emulsion in the PVA (w/o/w), followed by evaporation.

Whichever route of synthesis is adopted and whatever materials are used the microspheres may be loaded with substances of interest, such as pharmaceutically active substances as herein defined.

Microparticles, for example microbeads, may also be loaded by covalently attaching a pharmaceutical substance of interest to the bead using established chemical techniques.

Alternatively, for the controlled release, and/or immediate release, and/or sustained release of the pharmaceutically active substance, the polymeric material used to prepare the microparticles may include, but is not limited to, the following hydrophilic and/or lipophilic substances.

Natural or synthetic hydrophilic polymeric substances, can be used in the preparation of said microparticles which are biocompatible and/or biodegradable materials and pharmaceutically acceptable, e.g. polyvinylpyrrolidone in particular non-cross-linked polyvinylpyrrolidone (e.g. of molecular weight 30,000-400,000), hydroxypropylcellulose with a molecular weight of from 100,000 to 4,000,000, sodium carboxymethylcellulose (for example non-cross-linked, typical molecular weight 90,000-700,000), carboxymethylstarch, potassium methacrylate-divinylbenzene copolymer, hydroxypropylmethylcellulose with a molecular weight between 2,000 and 4,000,000, polyethyleneglycols of different molecular weight preferably between 200 and 15,000 (more preferably 1000-15000) and polyoxyethylenes of molecular weight up to 20,000,000 (more preferably 400,000-7,000,000), carboxyvinylpolymers, poloxamers (polyoxyethylene-polyoxypropylene copolymer), polyvinylalcohols, glucanes (glucans), carrageenans, scleroglucanes (scleroglucans), mannans, galactomannans, gellans, xanthans, alginic acid and derivatives (e.g. sodium or calcium alginate, propylene glycol alginate), polyaminoacids (e.g. gelatin), methyl vinyl ether/maleic anhydride copolymer, carboxymethylcellulose and derivatives (e.g. calcium carboxymethylcellulose), ethylcellulose, methylcellulose, starch and starch derivatives, alpha, beta or gamma cyclodextrin, and dextrin derivatives (e.g. dextrin) in general. The hydrophilic polymeric substance is therefore one which can be described as a controlled release polymer or a polymeric substance which is capable of achieving controlled release (CR).

More preferably for achieving advantageous controlled release of the active substance the hydrophilic polymeric substances may comprise one or more of the following: hydroxypropylcellulose with a molecular weight of from 100,000 to 4,000,000, hydroxypropylmethylcellulose (HPMC) with a molecular weight between 2,000 and 4,000,000 (more preferably between 10,000 and 1,500,000 molecular weight, still more preferably between 20,000 and 500,000 molecular weight, most preferably about 250,000 molecular weight), ethylcellulose or methylcellulose. The most preferred controlled release polymer is HPMC.

Hydrophilic polymeric substances such as sodium carboxymethylcellulose and/or calcium carboxymethylcellulose that act as viscosity-increasing agents/polymers may also be present

Thus, the hydrophilic polymeric substances may comprise sodium carboxymethylcellulose, carboxymethylcellulose or a derivative (e.g. calcium carboxymethylcellulose), hydroxypropylcellulose with a molecular weight of from 100,000 to 4,000,000, a carboxyvinylpolymer, a carrageenan, a xanthan, alginic acid or a derivative (e.g. sodium or calcium alginate, propylene glycol alginate), ethylcellulose, methylcellulose, dextrin and/or maltodextrin. The sodium carboxymethylcellulose (NaCMC) may be present in the form of non-cross-linked, molecular weight 90,000-700,000 NaCMC.

For all the polymers cited different types are commercially available characterised by different chemical, physical, solubility and gelification properties. In particular, as regards, hydroxypropylmethylcellulose various types with a different molecular weight (between 1,000 and 4,000,000, preferably from 2,000 to 4,000,000, even more preferably between 10,000 and 1,500,000 molecular weight, still more preferably between 20,000 and 500,000 molecular weight, most preferably about 250,000 molecular weight) can be used and with different degrees of substitution. Said types of hydroxypropylmethylcellulose have differentiated characteristics being mainly erodible or able to be gelled, depending on the viscosity and the degrees of substitution (D.S.) present in the polymeric chain. Gellable HPMCs (e.g. Methocel K grades) are preferable to erodible HPMCs (e.g Methocel E grades). The polyethyleneglycols and polyoxyethylenes show identical behaviour: in fact, different hydrophilic and gelification properties correspond to different molecular weights.

The molecular weight of polymers and the 2% viscosity of polymers can be directly correlated (“METHOCEL™ in Aqueous Systems for Tablet Coating”, page 12, published by The Dow Chemical Company—located on the world wide web at “dow”dot“com”, where “dot” is a period—METHOCEL™ is a trademark of The Dow Chemical Company) where viscosity of a polymer is defined as viscosity of a 2% aqueous solution at 20° C. measured as mPa. seconds. Viscosity is measured in Pascal seconds (SI units) or in poise (c.g.s. units), where 1 centipoise=10⁻Pa·sec. So for example, METHOCEL™ K100M has an approximate molecular weight of 246,000 and a corresponding 2% viscosity of 100,000 mPa·sec (based on an average viscosity of 80,000 to 120,000 mPa·sec.); METHOCEL™ K4M has an approximate molecular weight of 86,000 and a corresponding 2% viscosity of 4,000 mPa·sec; and METHOCEL™ K100LV has an approximate molecular weight of 27,000 and a corresponding 2% viscosity of 100 mPa·sec. For this reason, the preferred molecular weight ranges of the polymeric substances, for example the hydroxypropylmethylcellulose polymers can also be defined in terms of viscosity.

One preferred viscosity range for the hydroxypropylmethylcellulose polymers as defined above may be in the range of from 50 to 150,000 mPa·sec, suitably 80,000 to 120,000 mPa·sec (e.g. K100M).

In an alternative embodiment, in order to obtain a faster release rate, the viscosity range for the hydroxypropylmethylcellulose polymers in the active and/or barrier layer(s) may be in the range of from 50 to 25,000 mPa·sec (including Methocels K4M, K15M, K100LV). In this embodiment, preferably some or all of the HPMC polymers have a viscosity in the range of from 1000 to 25,000 mPa·sec (including Methocels K4M & K1SM but not K100LV or K100M). More preferably, HPMC polymers having a viscosity in the range of from 1000 to 25,000 mPa·sec are present in the active or barrier layer in a percentage of from 5 to 50% by weight of the active or barrier layer.

Lipophilic substances may also be utilised if desired, for example natural fats (coconut, soya, cocoa) as such or totally or partially hydrogenated, beeswax, polyethoxylated beeswax, mono-, bi- and tri-substituted glycerides, glyceryl palmitostearate, glyceryl behenate (glyceryl tribehenate commercially known as Compritrol 888), diethyleneglycol palmitostearate, polyethyleneglycol stearate, polyethyleneglycol palmitostearate, polyoxyethylene-glycol palmitostearate, glyceryl monopalmitostearate, cetyl palmitate, mono- or di-glyceryl behenate (glyceryl mono-behenate or glyceryl di-behenate), fatty alcohols associated with polyethoxylate fatty alcohols, cetyl alcohol, stearic acid, saturated or unsaturated fatty acids and their hydrogenated derivatives, hydrogenated castor oil and lipophilic substances in general.

The microparticles the invention may be prepared blending, milling and/or grinding (or co-grinding) the active substance and the hydrophilic and/or lipophilic polymeric substance, followed by dry granulation or wet granulation. Appropriate binder or adjuvant substances may be used, if desired.

Dry Granulation is granulation by compression of powders by either slugging or roller compaction. It is essentially a densification process. Slugging is where a crude compact (slug) is produced to a set weight/thickness for a given diameter of slug. These slugs are then reduced by either grating or commuting mill to produce granules of the required particle size/range.

Roller compaction or Chilsonating is where a powder mix is forced via an auger between 2 rollers (which can be smooth or grooved). Compaction of this material is controlled by the feed rate to the rollers and the hydraulic force of the rollers being pushed together. The resulting compact (called a ribbon or strip) is then reduced by either grating or commuting mill to produce granules of the required particle size/range.

Where dry granulation is used, the adjuvants often differ slightly compared to wet granulation. For example, instead of lactose monohydrate (often used in wet granulation), one preferably uses spray-dried lactose preferably containing amorphous lactose (e.g. Fast-Flo lactose, Seppic, Paris, France).

Wet Granulation is the a widely used granulation technique, and involves powder densification and/or agglomeration by the incorporation of a granulation fluid/medium to the powder mix. Wet granulation can be aqueous-based or solvent-based, e.g. based on organic solvents. Shear is dependent on the speed of the granulator paddle/blade through the powder. Various mixer designs are available, for example:
Wet High Shear, (rotating high shear forces (Fielder))
Wet Low Shear, (rotating low shear forces (Planetary mixer))
   Wet Low Shear Tumble, (spraying in to tumble mixer with/without intensifier bar)
   Extrusion, (Wet solids pushed through classified screen)
   Rotary Granulators, (Spheronisation, Marumerisation—spinning disk or walls of a vessel)
   Spray granulation in a fluidised Bed, or
   Spray dry granulation.

Methods for the production of microparticles of desired sizes are also known in the art. Such methods can involve the use of homogenisation or high shear forces.

Other routes of formulation may also be used, for example spray drying, lypophilisation etc.

The microparticles are of at least 0.5 μm average particle diameter, suitably in the range of from about 0.51 μm to 4.5 μm, 1.0 μm to 4.0 μm, 2.0 μm to 3.5 μm or 2.0 μm to 3.0 μm. Particle sizes can be measured by scanning electron microscopy or by confocal microscopy. The microparticles may also be referred to as microbeads or microspheres. Preferred particle size ranges may be from 1.5 μm to 3.5 μm, suitably 2.0 μm to 3.0 μm.

The pharmaceutically active substance may be any suitable drug or other biologically active substance, for example, protein, nucleic acid, carbohydrate, glycosaminoglycan, proteoglycan, peptide nucleic acid, and in addition including radio-isotopes and/or radio-isotope labelled molecules.

Nucleic acid includes, DNA, cDNA, RNA, mRNA, siRNA, ribozymes, aptamers etc. The nucleic acid may be present as an oligonucleotide, and may be sense or antisense. The term “nucleic acid” therefore includes oligonucleotides, polynucleotides or fragments or derivatives thereof.

Proteins include, hormones, cytokines, receptors, antibodies etc. The term “protein” in this text means, in general terms, a plurality of amino acid residues joined together by peptide bonds. It is used interchangeably and means the same as peptide, oligopeptide, oligomer or polypeptide, and includes glycoproteins and derivatives thereof. The term “protein” is also intended to include fragments, analogues and derivatives of a protein wherein the fragment, analogue or derivative retains essentially the same biological activity or function as a reference protein. A protein according to the invention may have additional N-terminal and/or C-terminal amino acid sequences. Such sequences can also be modified, such as for example by glycosylation.

Examples of such proteins may include, but is not limited to, a growth factor (e.g. TGFβ, epidermal growth factor (EGF), platelet derived growth factor (PDGF), nerve growth factor (NGF), brian-derived growth factor (BDNF), neurotrophin-3 (NT-3), neurotrophin-4 (NT-4), neurotrophin-5 (NT-5), neurotrophin-4/5 (NT-4/5), glial cell line-derived neurotrophic factor (GDNF), ciliary neurotrophic factor (CNTF), colony stimulating factor (CSF), hepatocyte growth factor, insulin-like growth factor, placenta growth factor); differentiation factor; cytokine e.g. interleukin, (e.g. IL1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20 or -IL21, either α or β), interferon (e.g. IFN-α, IFN-β and IFN-γ), tumour necrosis factor (TNF), IFN-γ inducing factor (IGIF), bone morphogenetic protein (BMP); chemokine (e.g. MIPs (Macrophage Inflammatory Proteins) e.g. MIP1α and MIP1β; MCPs (Monocyte Chemotactic Proteins) e.g. MCP1, 2 or 3; RANTES (regulated upon activation normal T-cell expressed and secreted)); trophic factors; cytokine inhibitors; cytokine receptors; free-radical scavenging enzymes e.g. superoxide dismutase or catalase; pro-drug converting enzymes (e.g. angiotensin converting enzyme, deaminases, dehydrogenases, reductases, kinases and phosphatases); peptide mimetics; protease inhibitors; tissue inhibitor of metalloproteinase sub classes (TIMPS) and serpins (inhibitors of serine proteases); peptide hormones include, insulin, growth hormone, melanocortin, adrenocorticotrophin hormone (ACTH).

As used herein “peptide mimetics” includes, but is not limited to, agents having a desired peptide backbone conformation embedded into a non-peptide skeleton which holds the peptide in a particular conformation. Peptide mimetics, which do not have some of the drawbacks of peptides, are of interest in those cases where peptides are not suitable in medicine.

Peptide mimetics may comprise a peptide backbone which is of the L or D conformation. Examples of peptides mimetics include melanocortin, adrenocorticotrophin hormone (ACTH) and other peptide mimetic agents which play a role in the central nervous system, endocrine system in signal transduction and in infection and immunity.

Alternatively, the protein may be a bacterial toxin, such as for example a botulinum toxin derived from a Clostridium botulinum species. The botulinum toxins include Type A, B, Cl, D, E, F, and G. Suitably the botulinum toxin is Type A (also known as Botox®). Other bacterial toxins may be employed such as those derived from Salmonella, Bifidobacterium, Diphtheria or Pseudomonas species.

Other toxins may be used which have effects on nerve cells, such as for example excitotoxins or metabolic toxins. Excitotoxins include ibotenic and/or quinolinic acid and metabolic toxins include nitropropionic acid and/or malonic acids. Still further suitable toxins include 6′-hydroxydopamine (6-OHDA) and n-methyl-4-phenyl-1,2,3,6-tetra hydropiridine (MPTP).

The pharmaceutically active substance may be a drug acting on the cardiovascular system, such as anti-arrhythmics, cardiac stimulants, vasodilators, calcium antagonists, anti-hypertensives, for example anti-adrenergic substances of central and peripheral action or substances acting on the arteriolar musculature, analgesic substances, substances acting on the renin-angiotensin system, anti-hypertensives and diuretics in association, anti-Parkinson's Disease agents, diuretics and drugs for the treatment of Alzheimer's disease, anti-histamines and/or anti-asthmatics.

Examples of active substances which may be used in such pharmaceutical forms are: propranolol, atenolol, pindolol, ropinirole, prazosin, ramipril, spirapril; spironolactone, metipranolol, molsidomine, moxonidina, nadolol, nadoxolol, levodopa, metoprolol, timolol, or dopamine. In the treatment of Parkinson's Disease, levodopa may be co-administered with an inhibitor of catechol-O-methyl transferase, such as carbidopa or benseazide.

Analgesic substances include, but are not limited to, steroidal anti-inflammatory drugs, opioid analgesics, and non-steroidal anti-inflammatory drugs (NSAIDs). The analgesic substance may be a non-steroidal anti-inflammatory drug (NSAID), such as acetyl salicylic acid, salicylic acid, indomethacin, ibuprofen, naproxen, naproxen sodium, flubiprofen, indoprofen, ketoprofen, piroxicam, diclofenac, diclofenac sodium, etodolac, ketorolac, or the pharmaceutically acceptable salts and/or derivatives or mixtures thereof.

Other suitable analgesic substances include, but are not limited to opioid analgesics such as alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tramadol, tilidine and pharmaceutically acceptable salts and/or derivatives or mixtures thereof.

Anti-hypertensive drugs may include, diltiazem, trapidil, urapidil, benziodarone, dipiridamole (dipyridamole), lidoflazine, naphthydrofuryl oxalate, perhexeline maleate, oxyfedrine hydrochloride. Anti-histamines and/or anti-asthmatics may include ephedrine, terfenadine, theophylline or chlorpheniramine.

The microparticles may comprise one or more pharmaceutically active substances, or alternatively, different populations of microparticles prepared with different pharmaceutically active substances may be admixed prior to use, or during use.

The microparticles may also comprise a fluorescent protein such as GFP, or a fluorescent dye such as Dragon Green.

According to a second aspect of the invention, there is provided a method for the treatment of a disease or condition of the nervous system comprising the administration of a microparticle of average particle diameter 0.5 μm containing a pharmaceutically active substance effective to treat said disease or condition of the nervous system. Such methods extend to and include the use of a microparticle of average particle diameter 0.5 μm containing a pharmaceutically active substance in the preparation of a medicament for the treatment or prophylaxis of a disease or condition of the nervous system.

As noted above, one or more pharmaceutically active substances may be formulated in this manner for use in accordance with the present invention. Accordingly, the present invention also comprises a kit of parts comprising individual preparations of microparticles formulated with different pharmaceutically active substances for separate, simultaneous or sequential administration. Preferably such kits additionally comprise instructions for use.

Diseases of the nervous system include, but are not limited to, diseases such as Cancer, Motor Neuron Disease (MND), Huntingdon's disease, Parkinson's disease and Alzheimer's disease. Cancer includes both sarcomas and carcinomas. Other non-malignant tumours of a benign origin, i.e. not malignant, are also included.

Familial cases of MND (also known as Amyotrophic Lateral Sclerosis, ALS) have been shown to involve mutations in Superoxide Dismutase 1 (SOD 1).

As defined above, nerve cells or neurons include neuronal cell lines, such as PC12 cells, primary neuronal cell cultures, dorsal root ganglion cells, motor neurons and cortical neurons.

Nerve fibres or nerves pass signals from the cells, tissues and organs of the body to the brain and vice versa. The nervous tissue of the body includes the brain, spinal cord and other specialised nervous tissues.

According to a third aspect of the invention, there is provided the use of a microparticle of average particle diameter 0.5 μm containing a pharmaceutically active substance to treat a disease or condition of the nervous system.

According to a fourth aspect of the invention, there is provided a unit dosage form of a pharmaceutical composition for the treatment of a disease or condition of the nervous system in which said pharmaceutical composition comprises a plurality of microparticles of average particle diameter 0.51 μm containing a pharmaceutically active substance to treat the said disease or condition of the nervous system.

According to a fifth aspect of the invention, there is provided a pharmaceutical composition comprising a plurality of microparticles of average particle diameter 0.5 μm containing a pharmaceutically active substance to treat a disease or condition of the nervous system.

Compositions or dosage forms according to the present invention may be administered by any convenient route, for example, intra-muscular, intrathecal, intraocular, intranasal, oral etc. It may be particularly suitable to administer the composition or dosage form neuronally, namely by direct administration to a nerve, a nerve cell or nervous tissue or organ for peripheral transport into the nervous system, i.e. into the neuron to be treated.

The compositions or dosage forms may formulated in accordance with standard pharmaceutical practice with appropriate diluents and/or carriers, or adapted for a particular route of administration.

Preferred features for the second and subsequent aspects of the invention are as for the first aspect mutatis mutandis.

### EXAMPLES

Materials and Methods

A list of the experimental models investigated in this study is shown in Table 1. All experiments were carried out simultaneously with relevant controls, which are detailed in Table 1. For time-lapse experiments using wild type rat motor neurons, neighbouring non-apoptotic neurons have been used as internal control (Table 1).

Morphological Tissue Studies

Ethical committee approval was obtained for the use of human tissue, derived from the archive of the Institute of Pathology or the Institute of Neurology, Queens Square. Animals were maintained in accordance with Home Office (UK) regulations. Tissues for morphological studies were fixed in paraformaldehyde (PFA)(animal tissues) or formalin (human and animal samples) and included in paraffin wax. 3 μm sections were cut and slide mounted on APES-coated slides. Sections were deparaffinised in xylene prior to rehydration for staining. Standard haematoxylin and eosin or Luxol fast blue/Glees and Marsland techniques were used in human tissues. Immunohistochemistry was performed according to the avidin-biotin-peroxidase complex (ABC) method employing the Vectastain Elite ABC kit (Vector, UK), where a streptavidin-biotin-immunoperoxidase system was used, using the Stept-ABC kit (DakoCytomation, UK). Endogenous peroxidase activity was blocked by immersion in 3% H₂O₂ for 15 min, followed by thorough washing. The peroxidase reaction was developed with 0.5 mg/ml 3,3′-diaminobenzidine and 0.02% hydrogen peroxide. Tissue sections were counterstained in haematoxylin, dehydrated and mounted in DPX (BDH, UK). Slides were observed by light microscopy (LM) with an IM35 microscope (Carl Zeiss, D).

Rat Embryo Motor Neuron Cultures and Analysis

Primary motor neuron cultures (Lalli et al. 2002) at DIV 7-8 were incubated with 2.5×10⁷ microspheres/ml in complete medium supplemented with 30 mM HEPES-NaOH pH 7.4 for 18 h at 36° C. Cells were then fixed in 4% PFA, 20% sucrose, permeabilised with 0.1% Triton X-100 in phosphate buffered saline (PBS), blocked in 2% bovine serum albumin, 10% normal goat serum, 0.2% glycine, 0.25% fish skin gelatin in PBS and incubated first with anti-p-tubulin antibody (mouse monoclonal, clone KMX-1, Boehringer Mannheim, D), then with Alexa488 Fluor conjugated goat anti-mouse antibody (Molecular Probes, CA) in blocking solution. Fluorescent staining was visualised using a Zeiss LSM 510 confocal microscope equipped with a 63×, 1.4 NA Plan Apochromat, Phase 3 oil-immersion objective (Zeiss, D).

For time-lapse microscopy, motor neurons at DIV1 and DIV8 were incubated as described above and imaged with a Nikon Diaphot 200 inverted low-light microscope equipped with a 20× or 40× DL objective using a Hamamatsu Orcal digital camera controlled by Kinetic AQMAdvance6 software (Kinetic Imaging, UK).

DRG Cell Cultures

Dorsal root ganglia (DRGs) from 1′-day chick embryos were cultured in Eagle's basal medium containing 10% horse serum, 1% L-glutamine, 1% cell culture tested penicillin/streptomycin solution and snake venom derived Nerve Growth Factor (NGF) (Sigma, UK). They were maintained at 37° C. either on collagen-coated glass or Melinex (Sigma, UK) substrates in an atmosphere of 10% CO₂. The explants were re-fed at intervals of 72 h and maintained in vitro for periods ranging between 5 and 10 days. After 3-4 days, cultures with many outgrowing axon bundles were re-fed. Half were confronted with microspheres (see below), the other half used as controls. DRGs were then maintained for a further 24 h in culture. These were studied by light microscopy, transmission and scanning electron microscopy. Cells were fixed in 4% PFA for 15 min and then washed in TBS, and blocked with 10% donkey serum for 30 min. Cells were incubated with primary antibody for 1 h, washed and then incubated with 1:400 dilution of donkey anti-mouse FITC for 1 h, followed by washing. Cells were mounted using 3:1 PBS:glycerol.

Mouse Cerebral Cortex Cultures

Cells derived from the hemispheres of an adult BALB/c mouse brain treated with trypsin were cultured in Eagle's basal medium containing 10% horse serum, 1% L-glutamine, 1% cell culture tested penicillin/streptomycin solution (Sigma, UK) and Brain Derived Growth Factor (BDNF) (Sigma, UK) and maintained at 37° C. on collagen-coated glass coverslips (Sigma, UK) in an atmosphere of 10% CO₂. The explants were fed at intervals of 72 h and kept in vitro for 5-10 d. After 3-4 d, differentiated cultures were either confronted with Dynabead microspheres (see below) or used as controls, and maintained for a further 24 h in culture.

Microsphere Studies

Dynabeads (Dynal, N) are uniform, magnetic beads composed of highly cross-linked polystyrene. The microspheres are non-toxic and chemically stable under standard in vitro culture conditions. M-280 particles (2.8 μm average diameter) were vortexed in 0.1 M sodium phosphate buffer, pH 7.4, incubated for 10 min and then isolated by a standard magnet according to the manufacturers instructions. Beads were then carefully resuspended in 0.1 M sodium phosphate buffer to give a concentration of approximately 109 beads/ml. 2.5 μl of beads was added to each set of cells and left for 24 h. Beads were generally used uncoated, but in a parallel series of experiments using dorsal root ganglion neurons, beads were coated with fas ligand (Sigma, UK) prior to use.

Electron Microscopy

Rat spinal cord motor neurons grown on glass coverslips were washed three times in Neurobasal medium without serum and incubated for 15 min at 37° C. to wash out residual transferrin. Coverslips were then incubated with mouse monoclonal anti-Thy1 antibodies (Ox7 clone) directly conjugated to 10 nm gold particles (Odorizzi et al., 1996) and with 20 μg/ml human transferrin-horseradish peroxidase (Tf-HRP) conjugate (Hopkins et al., 2000) at 37° C. for 30 min. To induce formation of large polyvalent protein complexes, gold-conjugated antibodies and Tf-HRP were first mixed together before being diluted in Neurobasal medium. After incubation, cells were washed three times in Neurobasal medium and fixed at room temperature for 15 min with 2% PFA/1.5% glutaraldehyde in 100 mM sodium cacodylate, pH 7.5. Fixed cells were then treated with diaminobenzidine to crosslink HRP. All cells were then post-fixed with 1% osmium tetroxide/1.5% potassium ferricyanide, treated with tannic acid before dehydration as described previously (Hopkins et al., 2000) and embedded on Epon stubbs. The coverslips were removed by immersion in liquid nitrogen. Cells were sectioned en face and 60 nm sections were stained with lead citrate, and viewed in a Philips CM12 electron microscope.

DRG culture coverslips with adherent monolayers were washed briefly in phosphate-buffered saline, then fixed in 2.5% buffered glutaraldehyde (pH 7.4) for 1 h. Cells were then rinsed with distilled water and dehydrated in a series of graded ethanol solutions. Specimens were critically point dried, spatter coated with gold, and finally stored under vacuum until ready for observation with a JEOL scanning electron microscope.

India Ink Method

Sections were examined following India ink injection into the caudo-putamen of adult rats as described in detail in Zhang et al. (1992). Briefly, a total of 41 young adult Wister rats, approximately 150 g in weight and of both sexes, were used in this study. The animals were anaesthetised with ether and placed in a stereotactic frame. A 30-gauge needle was inserted into the left cerebral hemisphere through a burr hole at one of two sites: (a) 2.5 mm lateral to the bregma to a depth of 5.5 mm, this site was calculated for injection into the caudo-putamen; (b) 4 mm lateral to the bregma and 4.5 mm in depth, these co-ordinates were calculated for injection into the white matter. India ink suspensions (1 or 2 μl) were injected over a period of several minutes into the selected site using a micrometre attachment. Except for those animals killed within the first hour after injection, all animals were allowed to recover from the anesthetic and none showed any sign of neurological deficit. Animals were subsequently anaesthetised and perfused with 10% buffered formalin. Animals were killed at regular intervals between 5 min and 2 years after injection.

Coronal slices of cerebral hemispheres were dehydrated and embedded in paraffin. Paraffin sections (5 μm) were stained with haematotoxylin and eosin (H&E).

### REFERENCES

Hopkins, C. Gibson, A. Stinchcombe, J. Futter, C. Methods Enzymol. 327, 35 (2000).
Lalli, G. et al., J. Cell Biol. 156, 233 (2002).
Odorizzi, G. et al., J. Cell Biol. 135, 139 (1996).
Zhang, T. et al., Acta Neuropathol. (Berl) 83, 233 (1992).
   Results:

### Example 1

### Bulk Material Phagocytosis

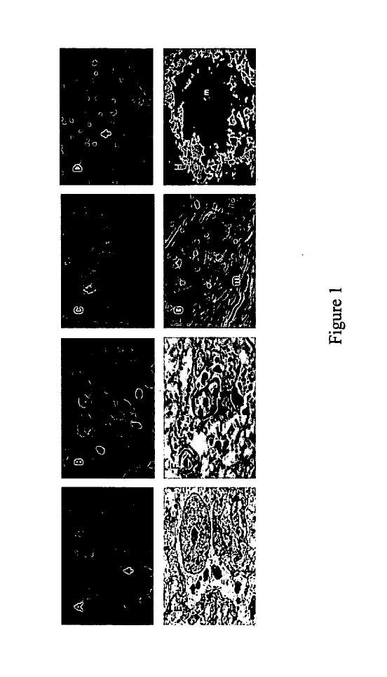

Bulk debris was observed being ingested by neurons (FIG. 1). This phenomenon was further analysed as follows using a range of in vitro and in vivo models (listed in Table 1), examining the physiological consequences of this process for neurons.

### Example 2

### India Ink Particle Phagocytosis

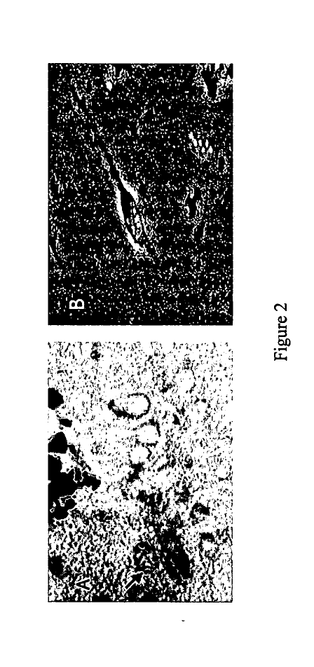

Following the injection of India ink into the caudo-putamen of rat brains in vivo, India ink particles of 0.5 microns and above were observed as inclusions in neurons of the basal ganglia in histological preparations (FIG. 2A) from 11 days post injection. Similar findings were seen at a range of time points, up to 2 years after injection, in the absence of significant neurological deficit or evidence of associated neuronal cytopathology.

### Example 3

### Studies on Phagocytosis in Cortical Neurons

In vitro studies of cultured mouse adult cortical neurons, as well as adjacent non-neuronal cells, readily took up administered 2.8 micron microspheres, with several microspheres detected in a single neuron (FIG. 2B).

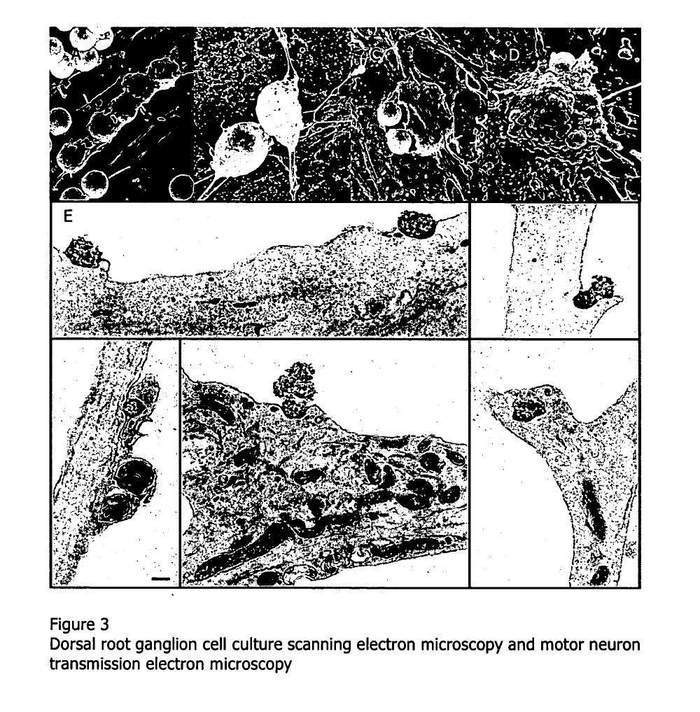

Cultured chick embryo DRG neurons, as well as adjacent non-neuronal cells, readily took up administered DRG debris as well as microspheres (FIG. 3A,B), with up to 7 microspheres detected in a single neuron (FIG. 3C). Cell debris appeared to have no deleterious effects on neuronal survival. It was noted that apoptosis was more frequent in cultures with DRG neurons containing microspheres, compared with control cultures (FIG. 3D). No differences were seen in uptake or cell survival between uncoated microspheres and those coated with fas ligand antigen.

Transmission electron microscopy of rat spinal motor neurons showed that the adhesion and uptake of large labelled protein aggregates (FIG. 3E) occurred both at the level of the soma and of the neurite. Uptake was not associated with morphologically distinct membrane specialisation, such as synaptic sites or axonal bundles.

### Example 4

### Studies on Phagocytosis in Spinal Cord Neurons

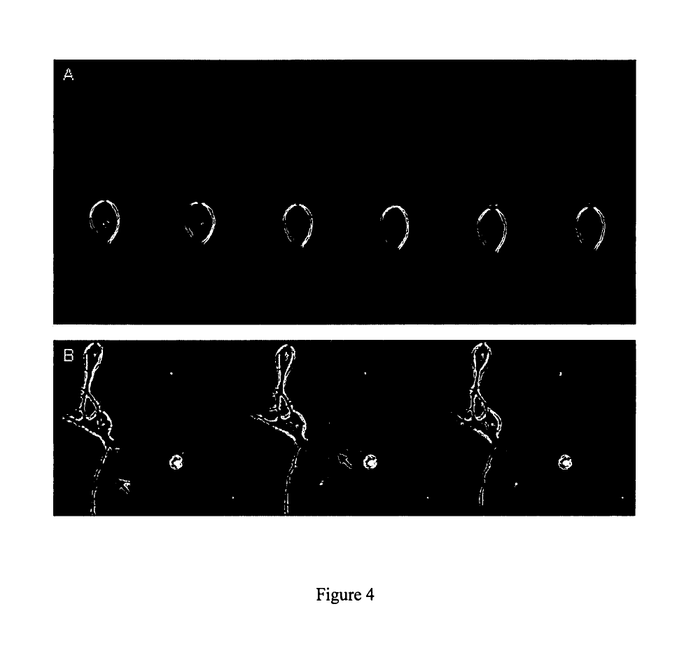

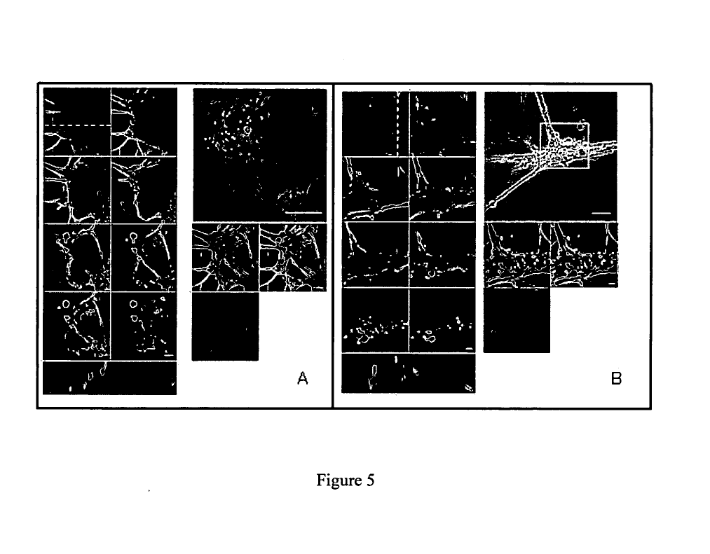

Time-lapse video microscopy of rat spinal cord neurons demonstrated that in control cultures, large volume debris, with an apparent size over three times the diameter of the neuronal process, could be rapidly transported toward the soma (FIG. 4A), suggesting that the functional integrity of neurites could be retained despite marked distortion of their usual diameter. Following death of cultured spinal cord neurons by apoptosis, an occasional feature in such control cultures, processes of adjacent cells were noted to take up the resultant debris, targeting this material to the cell body (FIG. 4B). The phagocytic activity of spinal motor neurons was not limited to cellular remnants, but extended to foreign bodies, such as polymeric microspheres which were seen to be taken up by occasional neurons when added to cultures. Confocal microscopy confirmed the presence of ingested microspheres within the neuronal cell body and showed instances of cell death associated with the uptake of microspheres (FIG. 5A,B).

In studies of human cerebral cortex, adjacent to areas of old cerebral haemorrhage, both macrophages (siderophages) and occasional cortical neurons are noted to contain granular debris, confirmed as iron-containing inclusions by the use of the Perl's Prussian blue reaction on histochemistry. This is a landmark of old cerebral haemorrhage well known by neuropathologists. Human cerebral neurons from a case of supefficial siderosis were also noted to contain large iron granules (FIG. 1F), suggesting that neurons, as well as macrophages, may play a role in the clearance of iron-containing siderotic debris.

This study therefore demonstrates that all types of neurons examined so far are phagocytic, with the ability to ingest extracellular material up to 2.8 microns in diameter. It has also been demonstrated that intraneuronal inclusions may originate from phagocytosis of extracellular material and that debris can be transported to the neuronal soma and that phagocytosed bodies can persist within neurons, or be associated with cell death.

TABLE 1
List of models examined and methods employed.

| Neuronal | | | In vivo/ | | | |
|---|---|---|---|---|---|---|
| type | Species | Stage | vitro | Type of debris | Control | Methods of analysis |
| Spinal | Mouse | E18 | In vivo | Degenerate | Wild type | Light microscopy |
| motor | (Loa | P0 | | motor neuronal | animals | Immunohistochemistry |
| neuron | mutant) | | | debris | | Transmission EM |
| Spinal | Rat | E13 | In vitro | Microspheres | Internal | Time-lapse video |
| motor | | | | | | microscopy |
| neuron | | | | | | Confocal microscopy |
| Spinal | Rat | E13 | In vitro | Apoptotic motor | Internal | Time-lapse video |
| motor | | | | neuronal debris | | microscopy |
| neuron | | | | | | |
| Spinal | Rat | E13 | In vitro | Large | Internal | Transmission EM |
| motor | | | | polyvalent | | |
| neuron | | | | protein | | |
| | | | | complexes | | |
| Dorsal root | Chick | E11 | In vitro | Microspheres | Cultures | Phase contrast |
| ganglion | | | | | with no | microscopy |
| cell | | | | | added | Scanning EM |
| | | | | | microspheres | Immunohistochemistry |
| Dorsal root | Chick | E11 | In vitro | Cell debris | Cultures | Phase contrast |
| ganglion | | | | | with no | microscopy |
| cell | | | | | added debris | Immunohistochemistry |
| Dorsal root | Human | Adult | In vivo | Lipid debris | Normal | Light microscopy |
| ganglion | | | | | human DRG | Histochemistry |
| cell | | | | | | |
| Peripheral | Human | Adult | In vivo | Myelin | Human | Transmission EM during |
| nerve | | | | | peripheral | histopathological |
| | | | | | nerve | examination |
| Cerebral | Human | Adult | In vivo | Iron granules | Normal | Light microscopy |
| cortex | | | | | human | Histochemistry |
| | | | | | cortex | |
| Motor | Mouse | Adult | In vitro | Microspheres | Cultures | Light microscopy |
| Cortex | | | | | with no | |
| | | | | | added | |
| | | | | | spheres | |
| Caudate | Rat | Adult | In vivo | India ink | Normal rat | Light microscopy |
| and | | | | particles | caudate and | Interference contrast |
| putamen | | | | | putamen | microscopy |

EM = electron microscopy

Microsphere Fabrication:
Materials:

PLGA (Poly-L-lactide-co-glycolide) (Resomer® LG824) from Boehringer Ingelheim Methylene chloride (dichloromethane) from BDH

PVA from Sigma

FITC-BSA from Sigma

Tween 80 from Sigma

Method:

100 μl of FITC-BSA solution (20 mg of FITC-BSA in 100 μl of distilled water) is added to 2 ml of 0.01% PLGA solution (0.02 g PLGA in 2 ml methylene chloride) then mixed by probe sonication at 50 Watts for 1 minute to form an emulsion. 1 ml of this primary emulsion is extracted and put into 4 ml of 1% PVA solution and further sonicated at 50 Watts for 1 minute to form the secondary emulsion (w/o/w). This emulsion is transferred into 300 ml of 0.1% PVA solution (containing 2.0 g of Tween 80) and stirred with a magnetic stirrer for 3 hours until total evaporation of the organic solvent (methylene chloride). The microspheres are then collected by centrifugation (200 rpm for 5 minutes).

Note: The FITC-BSA step may be substituted with other proteins or molecules, for instance rhodamine B has been added to produce beads that are fluorescent in the red.

### Example 5

### Uptake of Microspheres by Neurons

Microspheres were prepared from polystyrene and loaded with dragon green fluorescence as above. The microspheres were cultured with dorsal root ganglion (DRG) neurons under the following conditions.

Cell Culture

1. Using standard laboratory strain adult rat.

2. Euthanasia in line with Home Office regulations (schedule 1).

3. Dorsal root ganglia removed and placed in F12 Hams solution and return to the laboratory.

4. Add 5 mls of trypsin to the chopped cortex and incubate at 37° C. for 10 minutes.

5. Add 5 mls of fetal calf serum.

6. Centrifuge for 10 minutes at a speed of 2000 rpm. Pour off the liquid leaving the cells in the test tube.

7. Resuspend the cells in 5 ml culture medium containing:
40 mls Eagles medium
L-glutamine 0.4 ml
Streptomycin penicillin 0.8 m
BDNF 1 ml
CNTF 2 mls
* Add fetal calf serum 4 mls to the serum batch test tube.

8. Place collagen coated coverslips on a drop of DMEM in a Petri dish and label.

9. Pipette the resuspended cells over the surface of the coverslides (12 coverslides).

10. Cells were incubated under standard culture conditions until beads were added.

11. Immunofluorescent demonstration of added materials was then carried out according to the standard protocol.

Immunofluorescence Analysis

1. wash cells in PBS, then fix in 4% Paraformaldehyde/20% Sucrose in PBS for 15 min at room temperature (RT)

2. wash cells twice in PBS

3. incubate in 50 mM NH₄Cl in PBS for 20 min at RT

4. wash cells in PBS

5. incubate in 0.1% Triton—X100 in PBS for 5 min at RT

6. wash cells in PBS

7. block with 2% BSA/10% normal goat serum/0.25% fish skin gelatine in PBS for 1 h at RT

8. add primary antibody, diluted in blocking solution, and incubate for 30 min at RT

9. wash three times 5 min in PBS

10. add secondary antibody, diluted in blocking solution, and incubate for 30 min at RT

11. wash three times 5 min in PBS

12. wash in H₂O

13. mount in Mowiol-488

All solutions need to be filtered. As a control for background staining, prepare a coverslip with secondary antibody staining only.

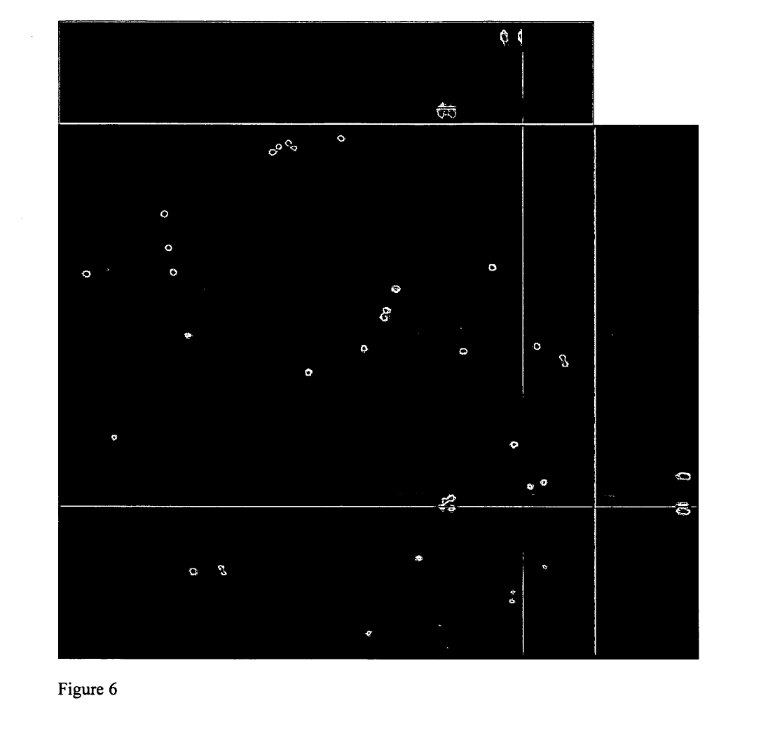

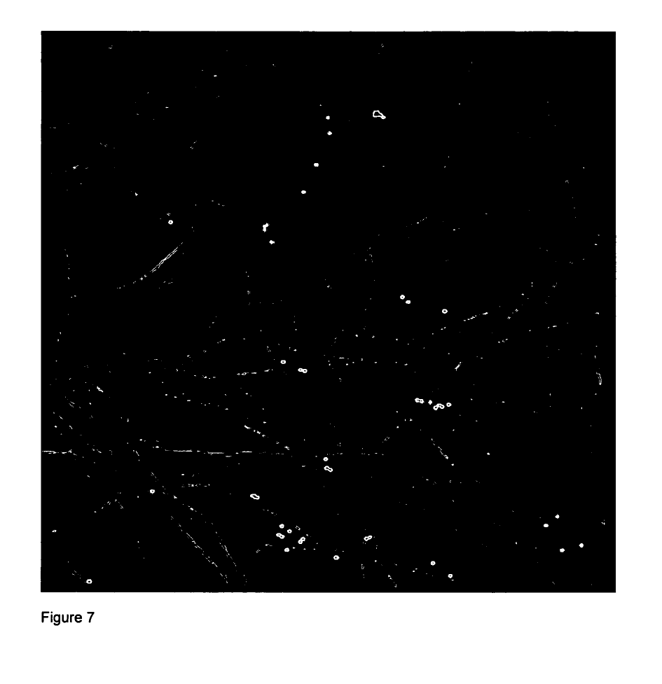

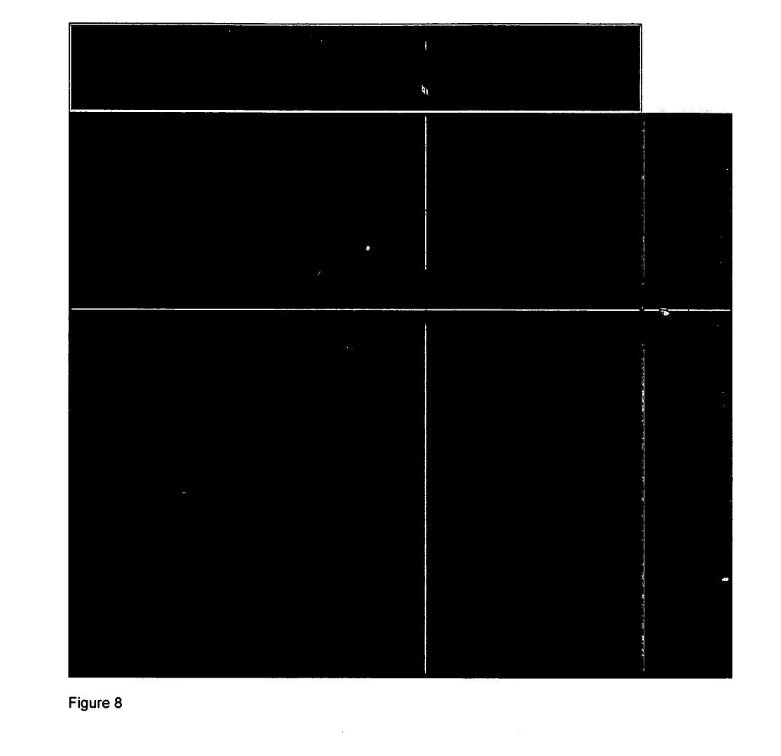

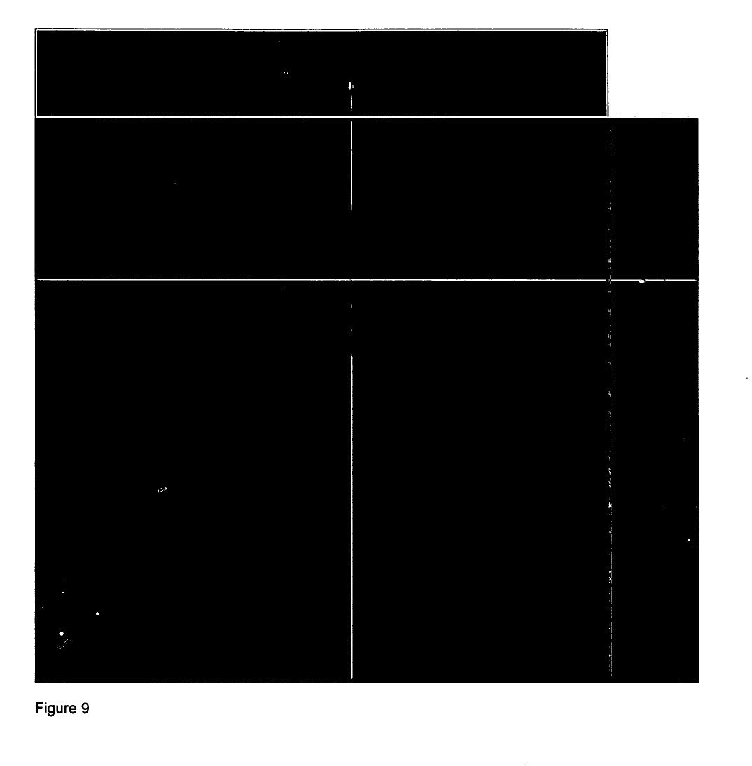

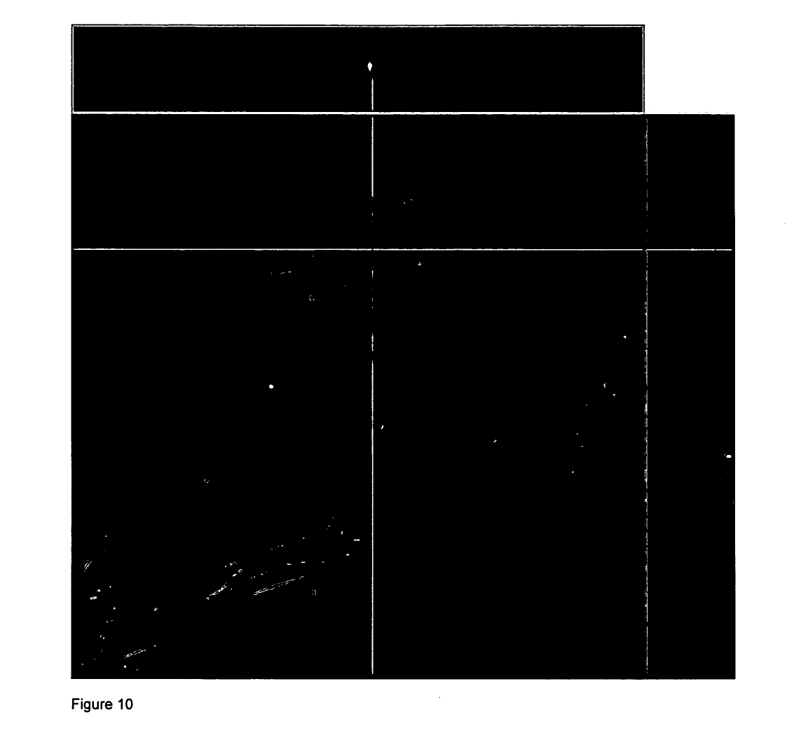

Photographs of neurons after incubation are shown in FIGS. 6, 7, 8, 9 and 10 which clearly indicate the uptake of microparticles into the neurons.

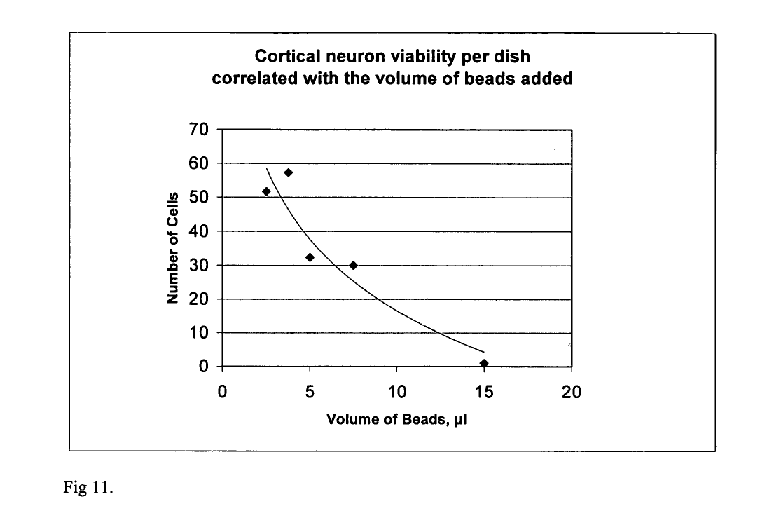

FIG. 11 shows a graph showing the addition of cortical neurons cultured (using the dorsal root ganglion cell protocol) with microbead particles added to cell culture dishes, demonstrating lack of toxicity at low concentrations.

### REFERENCES

1. Mellman, I. Ann. Rev. Cell Dev. Biol. 12, 575-625 (1996
2. Greenberg, S. & Grinstein, S. Curr. Opin. Immunol. 14, 136-145 (2002).
3. Underhill, D. M. & Ozinsky, A. Ann. Rev. Immunol. 20, 825-852 (2002).
4. Aderem, A. Cell 110, 5-8 (2002).
5. Gagnon, E. et al Cell 110, 119-131 (2002).
6. May, R. C. & Machesky, L. M. J. Cell. Sci. 114, 1061-1077 (2001).
7. Henson, P. Proc. Natl. Acad. Sci. USA 100, 6295-6296 (2003).
8. Egensperger, R., et al Dev. Brain Res. 97, 1-8 (1996).
9. Rosenbluth, J. & Wissig, S. L. Cell Biol. 23, 307-325 (1964)
10. Kristensson, K. Brain Res. Bull. 41, 327-333 (1996).
11. Smith, A. E. & Helenius, A. Science 304, 237-242 (2004).
12. Kaspar, B. K., et al Science 301, 839-842 (2003).
13. Boulanger, L. M., Shatz, C. J. Nat. Rev. Neurosci. 5, 521-531 (2004).
14. Riol-Blanco, L. et al. Eur. J. Immunol. 34, 108-118 (2004).
15. Eldred, G. E., Katz, M. L. Free. Radic. Biol. Med 10, 445-447 (1991).
16. Josephs, K. A. et al. Brain 126, 2291-2296 (2003).
17. Arima, K., et al, Acta Neuropathol. 100, 115-121 (2000).
18. Wada, M. et al. Neurosci. Lett. 356, 49-52 (2004).
19. Liao, L. et al. J. Biol. Chem. June 25 Epub (2005).
20. Ginsberg, S. D. et al. Ann. Neurol. 41, 200-209 (1997).
21. Johnston, J. A., et al J. Cell. Biol. 143, 1883-1898 (1998).
22. Ravikumar, B. et al. Hum. Mol. Genet. 12, 1107-1117 (2002).
23. Webb, J. L. et al. J. Biol. Chem. 278, 250-259 (2003).
24. Webb, J. L. et al. J. Biol. Chem. 278, 250-259 (2003).
25. Bruijn, L. I., et al Annu. Rev. Neurosci. 27, 723-749 (2004)
26. Ripps, H. Exp. Eye Res. 74, 327-336 (2002).
27. Cavanagh, J. B., et al Neurotoxicol. 11, 1-12 (1990).
28. Gatzinsky, K. P., et al Glia 20, 115-126 (1997).
29. Otter, A. & Blakemore, W. F. Acta Microbiol. Hung. 36, 125-131 (1989).
30. Khanolkar, V. R. Ind. Coun. Med. Res. Spec. Rep. Ser. 19, 1-33 (1951).
31. Yoshizumi, M. O. & Asbury, A. K. Acta Neuropath. 27, 1-10 (1974).
32. Gruenheid, S. & Finlay, B. B. Nature 422, 775-781 (2003).
33. Kohn, J. & Durham, H. D Neurotoxicology 14, 381-386 (1993)
34. Hafezparast, M. et al., Science 300, 808-812 (2003).
35. Revesz, T., et al J. R. Soc. Med. 81, 479-481 (1988).

1. A method for the delivery into a neuron of a microparticle of average particle diameter of at least 0.5 μm containing a pharmaceutically active substance, comprising the administration of said particle to said neuron.

2. A method as claimed in claim 1, in which the pharmaceutically active substance is a drug.

3. A method as claimed in claim 1, in which the pharmaceutically active substance is a protein, nucleic acid, carbohydrate, glycosaminoglycan, proteoglycan, or peptide nucleic acid.

4. A method for the treatment of a disease or condition of the nervous system comprising the administration of a microparticle of average particle diameter of at least 0.5 μm containing a pharmaceutically active substance effective to treat said disease or condition of the nervous system.

5. A method as claimed in claim 4, in which the disease of the nervous system is selected from the group consisting of Cancer, Motor Neuron Disease (MND), Parkinson's disease, Alzheimer's disease, and non-malignant tumours.

6. A microparticle of average particle diameter of at least 0.5 μm containing a pharmaceutically active substance for use in treatment of a disease or condition of the nervous system.

7. A unit dosage form of a pharmaceutical composition for the treatment of a disease or condition of the nervous system in which said pharmaceutical composition comprises a plurality of microparticles of average particle diameter of at least 0.5 μm containing a pharmaceutically active substance to treat said disease or condition of the nervous system.

8. A pharmaceutical composition comprising a plurality of microparticles of average particle diameter of at least 0.5 μm containing a pharmaceutically active substance to treat a disease or condition of the nervous system.
